(19) United States
(12) Patent Application Publication
(10) Pub. No.: US 2026/0276966 A1
(43) Pub. Date: Sep. 17, 2026

(54) MINIATURE MODULAR DETACHABLE IMAGE-OBSERVATION BASE DEVICE

(71) Applicant: National Taiwan University, Taipei (TW)

(72) Inventors: Chi-Kuang Sun, Taipei (TW); Chung-En Huang, Taipei (TW)

(21) Appl. No.: 19/172,269

(22) Filed: Apr. 7, 2025

(30) Foreign Application Priority Data
Mar. 13, 2025 (TW) 114109426

(51) Int. Cl. G02B 21/24; A61B 90/25; G02B 21/00

(52) U.S. Cl. CPC G02B 21/24; A61B 90/25; G02B 21/0028

(57) ABSTRACT

The present invention is a miniature modular detachable image-observation base device including a wearable optical base and a high-precision composite lens unit placed in the wearable optical base. The base's curved surface matches the natural curvature of a mouse's skull, allowing the composite lens unit to be firmly fixed to the mouse's head. The composite lens unit can be replaced with others to accommodate observation applications at different depths to not only overcome limitations of shallow brain regions but also provide a miniaturized gradient-index lens being slender and thus able to pierce the mouse's brain to reach deep brain regions thereof, so as to steadily perform functional imaging monitoring of neuronal activity in the deep brain regions during numerous sessions over prolonged periods.

### FIELD OF THE INVENTION

The present invention relates to a miniature modular detachable image-observation base device. More particularly, the present invention relates to a field of applications of neuroscience research and non-linear optical imaging, especially a device for conducting effective, prolonged, steady, and high-resolution observations of neuronal activity in a mouse's deep brain regions and the mouse's behavior.

### DESCRIPTION OF THE PRIOR ART

With existing miniaturized two-photon microscope technology, researchers can typically observe shallow brain regions, for example, the cortex and the hippocampus, only reaching depths of up to 2 millimeters (mm). However, with this technology, researchers are unable to observe and explore deeper brain regions, for example, substantia nigra, which is 4.3 mm deep and primarily responsible for the synthesis and release of dopamine. Deep brain regions play a key role in brain motor control and are also involved in reward mechanisms, motivation, behavior regulation, and learning. Therefore, deep brain regions are of vital importance in neuroscience due to their close connection with neurological disorders, such as Parkinson's disease and other movement disorders.

Since functional imaging of brain neurons has long been a major challenge to scientists, neuroimaging technology has rapidly advanced in recent years. However, conventional miniaturized two-photon microscope technology is effective for observing shallow brain regions (for example, the cortex and hippocampus) 2 mm deep but is not effective for imaging deeper brain regions (for example, the substantia nigra) 4.3 mm deep. Furthermore, prior art entails taking much time to fine-tune a mouse's head for optimal alignment, resulting in excessive experiment preparation time and reduced research efficiency. As a result, it is imperative to provide a detachable image-observation base device that overcomes the aforesaid drawbacks of the prior art, overcomes the 2 mm depth constraint, and achieves functional imaging monitoring of different brain regions by changing the base lens.

### BRIEF SUMMARY OF THE INVENTION

Therefore, the main purpose of the present invention is to overcome the aforesaid drawbacks of the prior art and provide a technology capable of high-resolution functional imaging of deep brain regions. This device overcomes the depth limitations of conventional microscope technology through proprietary optomechanical design, precisely aligned non-linear optical systems, and innovative electronic and software integration such that researchers can conduct steady, prolonged observations of neuronal activity in the deep brain regions and thereby conduct an in-depth study of the functions and pathology of these important brain regions.

To achieve the above purposes, the present invention is a miniature modular detachable image-observation base device, configured for use in high-resolution non-linear optical neuroimaging monitoring, comprising: a composite lens unit comprising a miniaturized gradient-index lens and a micro coupling lens; and a wearable optical base having a lower edge, the lower edge having a curved surface that matches the natural curvature of a mouse's skull, with a screw mounting sleeve disposed on the lateral side of the wearable optical base, allowing a screw to be mounted on the screw mounting sleeve to secure and align the base. A plurality of magnetic holes and a plurality of fixation points are disposed on the upper portion of the base to hold and secure a material for filling pores. The magnetic holes engagingly receive magnets to allow the magnets to be coupled to the base and fixed firmly thereto by magnetic attraction. The magnets correspond in number to the magnetic holes. The fixation points hold and secure a material for filling pores, receive screws for functioning as the fixation points in order to be fixed to the mouse's skull, and receive a filling material for enhancing fixation and reducing slight movement. The composite lens unit is detachably mounted inside the wearable optical base such that the wearable optical base fixes the composite lens unit firmly to a mouse's head. The composite lens unit can be replaced with others to accommodate observation applications at different depths to not only overcome limitations of shallow brain regions but also provide the miniaturized gradient-index lens being slender and thus able to pierce the mouse's brain to reach deep brain regions thereof, so as to steadily perform functional imaging monitoring of neuronal activity in the deep brain regions during numerous sessions over prolonged periods.

In the aforesaid embodiment of the present invention, the filling material consists of a dental material and a biocompatible colloid.

In the aforesaid embodiment of the present invention, the miniature modular detachable image-observation base device is less than 1 gram in weight.

In the aforesaid embodiment of the present invention, the wearable optical base has therein a lens receiving chamber for receiving the micro coupling lens, and the lens receiving chamber is in communication with an extending lens support pipe, allowing the miniaturized gradient-index lens to penetrate the extending lens support pipe to sink deep into the mouse's brain to reach the deep brain regions thereof.

In the aforesaid embodiment of the present invention, the extending lens support pipe is less than 2 mm in length.

In the aforesaid embodiment of the present invention, the miniature modular detachable image-observation base device further comprises a pulsed laser scanning module that functions as a miniaturized non-linear microscope configured to perform micron-scale resolution functional imaging monitoring of the deep brain regions.

In the aforesaid embodiment of the present invention, the miniaturized non-linear microscope is a miniaturized two-photon microscope.

In the aforesaid embodiment of the present invention, a plurality of slots are disposed on a lateral side of the wearable optical base and configured to be pressed by a human hand to facilitate the human hand's gripping the slots to increase points of force application in the course of removing the wearable optical base to facilitate the removal of the wearable optical base.

### BRIEF DESCRIPTION OF THE DRAWINGS

FIG. 1 is a lateral cross-sectional view of the device of the present invention;

FIG. 2 is a lateral view of a wearable optical base of the present invention;

FIG. 3 is a top view of the wearable optical base of the present invention;

FIG. 4 is a bottom view of the wearable optical base of the present invention;

FIG. 5A shows an image of a mouse wearing the wearable optical base in sleep mode according to the present invention;

FIG. 5B shows a neurofunctional image of deep brain regions of the mouse in sleep mode according to the present invention;

FIG. 5C shows an image from FIG. 5B captured at 16× playback speed;

FIG. 5D shows curves of calcium images generated through neurofunctional imaging analysis performed on the mouse in sleep mode over a long-time scale according to the present invention;

FIG. 5E shows an image of the mouse wearing the wearable optical base in movement mode according to the present invention;

FIG. 5F shows a neurofunctional image of the deep brain regions of the mouse in movement mode according to the present invention;

FIG. 5G shows an image from FIG. 5F captured at 8× playback speed;

FIG. 5H shows curves of calcium images generated through neurofunctional imaging analysis performed on the mouse in movement mode over a long-time scale according to the present invention;

FIG. 6A shows a dendrite functional image of the deep brain regions of the mouse in sleep mode according to the present invention;

FIG. 6B shows curves of calcium images generated through dendrite neurofunctional imaging analysis performed on the mouse in sleep mode over a long-time scale according to the present invention;

FIG. 6C shows a dendrite neurofunctional image of the deep brain regions of the mouse in movement mode according to the present invention; and

FIG. 6D shows curves of calcium images generated through dendrite neurofunctional imaging analysis performed on the mouse in movement mode over a long-time scale according to the present invention.

### DESCRIPTION OF THE PREFERRED EMBODIMENTS

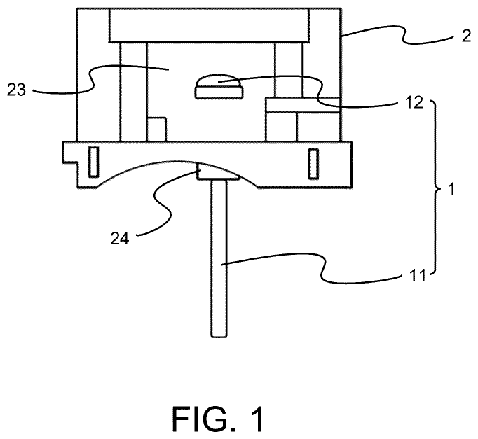

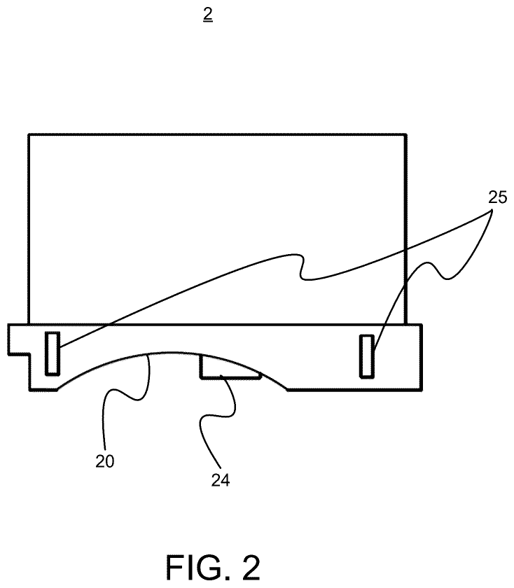

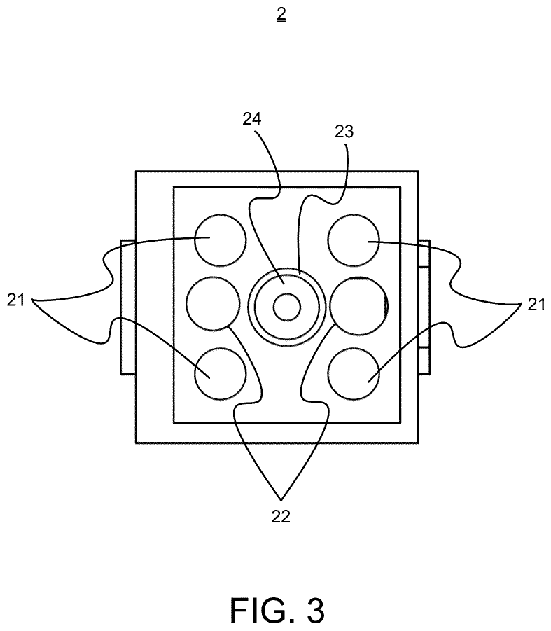

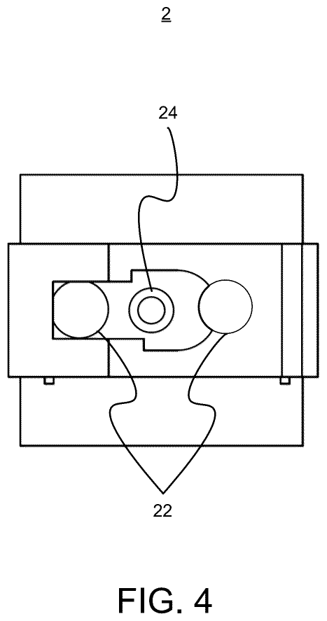

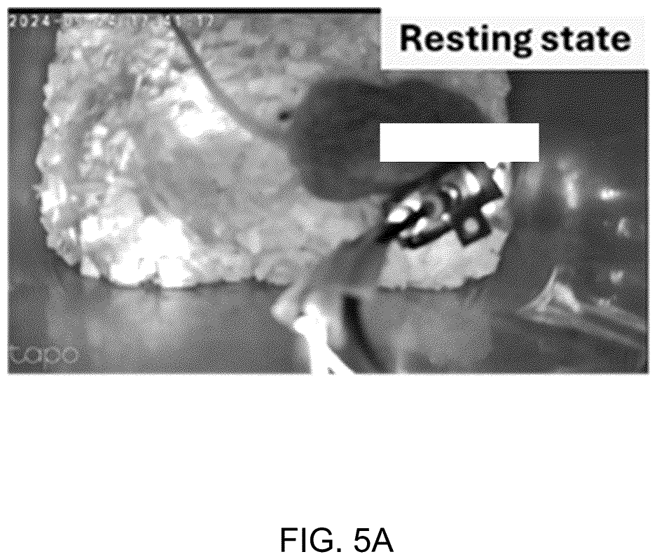

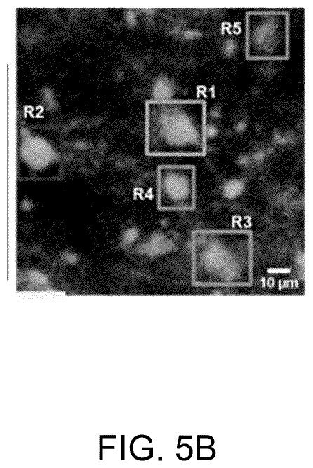

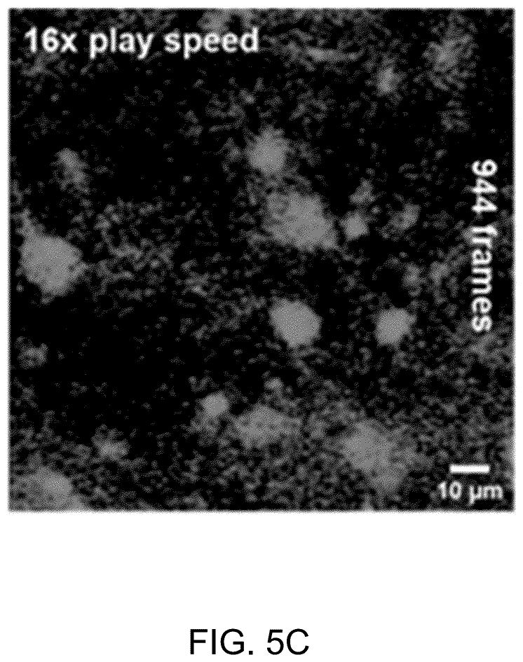

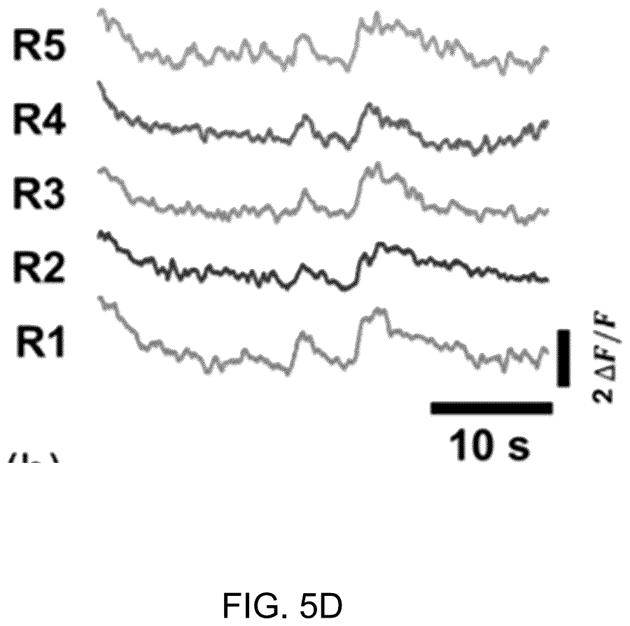

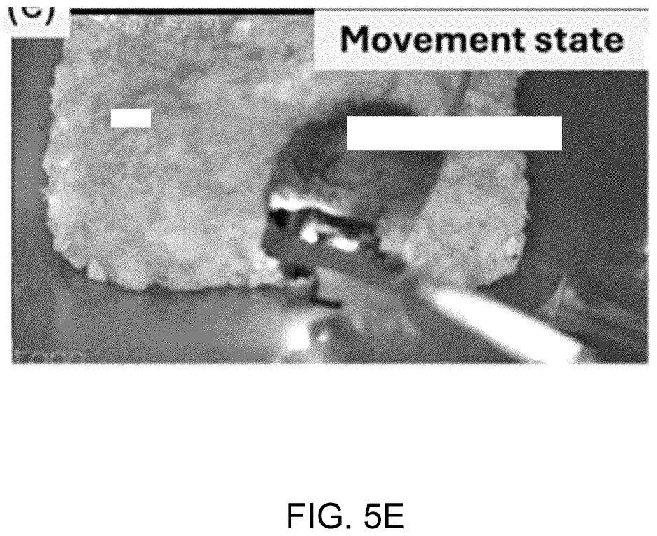

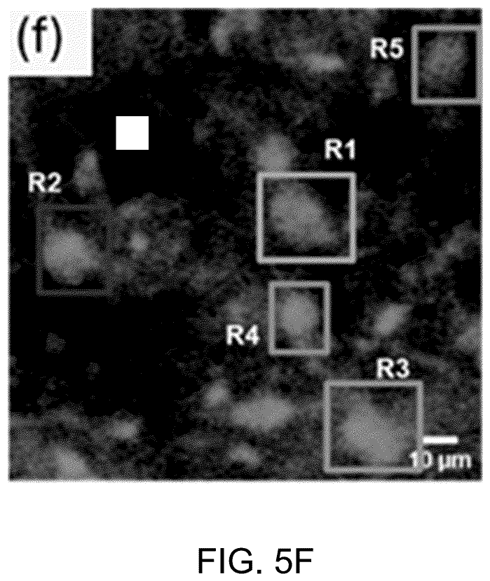

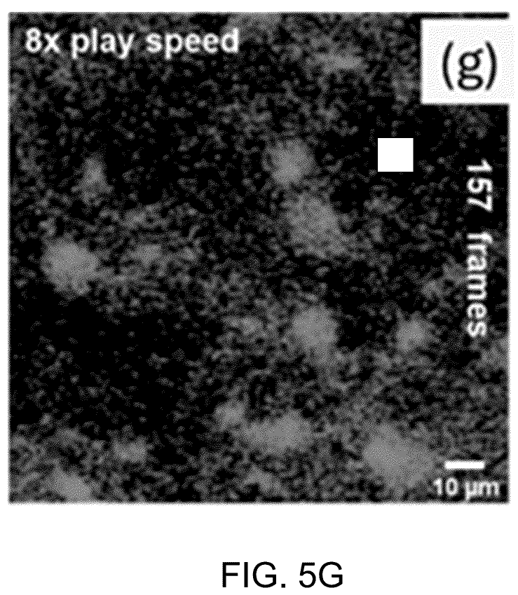

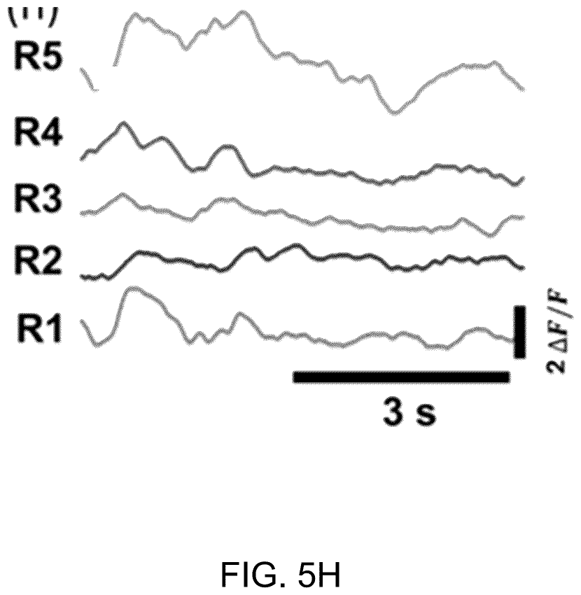

Referring to FIG. 1 through FIG. 6D, there are shown a lateral cross-sectional view of the device of the present invention, a lateral view of a wearable optical base of the present invention, a top view of the wearable optical base of the present invention, a bottom view of the wearable optical base of the present invention, an image of a mouse wearing the wearable optical base in sleep mode according to the present invention, a neurofunctional image of deep brain regions of the mouse in sleep mode according to the present invention, an image from FIG. 5B captured at 16× playback speed, curves of calcium images generated through neurofunctional imaging analysis performed on the mouse in sleep mode over a long time scale according to the present invention, an image of the mouse wearing the wearable optical base in movement mode according to the present invention, a neurofunctional image of the deep brain regions of the mouse in movement mode according to the present invention, an image from FIG. 5F captured at 8×playback speed, curves of calcium images generated through neurofunctional imaging analysis performed on the mouse in movement mode over a long time scale according to the present invention, a dendrite functional image of the deep brain regions of the mouse in sleep mode according to the present invention, curves of calcium images generated through dendrite neurofunctional imaging analysis performed on the mouse in sleep mode over a long time scale according to the present invention, a dendrite neurofunctional image of the deep brain regions of the mouse in movement mode according to the present invention, and curves of calcium images generated through dendrite neurofunctional imaging analysis performed on the mouse in movement mode over a long time scale according to the present invention, respectively. As shown in the diagrams, a miniature modular detachable image-observation base device 100 provided by the present invention is a multiple-lens assembly configuration platform firmly fixed to the mouse's head and configured for use in non-linear optical high-resolution neuroimaging monitoring. The device 100 comprises a composite lens unit 1 and a wearable optical base 2. This structural design combines functionality and biocompatibility, representing a significant innovation in neuroimaging equipment.

The composite lens unit 1 comprises a miniaturized gradient-index lens 11 and a micro coupling lens 12.

A lower edge of the wearable optical base 2 has a curved surface 20 that matches the natural curvature of a mouse's skull, ensuring installation stability and experimental accuracy. A screw mounting sleeve (not shown) is disposed on a lateral side of the wearable optical base 2. Screws are mounted on the screw mounting sleeve to facilitate the fixation and calibration of the wearable optical base 2. A plurality of magnetic holes 21 and a plurality of fixation points are disposed on the upper portion of the wearable optical base 2 to hold and secure a material for filling pores 22. The magnetic holes 21 engagingly receive magnets to allow the magnets to be coupled to the wearable optical base 2 and fixed firmly thereto by magnetic attraction. The magnets correspond in number to the magnetic holes 21. The fixation points hold and secure a material for filling pores 22, receive screws for functioning as the fixation points in order to be fixed to the mouse's skull, and receive a filling material for enhancing fixation and reducing slight movement. The composite lens unit 1 is detachably mounted inside the wearable optical base 2 such that the wearable optical base 2 fixes the composite lens unit 1 firmly to the mouse's head. The composite lens unit 1 can be replaced with others to accommodate observation applications at different depths to not only overcome limitations of shallow brain regions but also provide the miniaturized gradient-index lens being slender and thus able to pierce the mouse's brain to reach deep brain regions thereof. Furthermore, the wearable optical base 2 is less than 1 gram in weight and thus can be worn on the mouse's head for an extended period, allowing the functional imaging monitoring of neuronal activity in the deep brain regions to be steadily performed during numerous sessions over prolonged periods. Therefore, upon completion of the aforesaid process flow, a novel miniature modular detachable image-observation base device 100 is formed.

In a preferred specific embodiment of the present invention, the filling material consists of a dental material and a biocompatible colloid.

In a preferred specific embodiment of the present invention, the wearable optical base 2 has therein a lens receiving chamber 23 for receiving the micro coupling lens 12. The lens receiving chamber 23 is in communication with an extending lens support pipe 24. The miniaturized gradient-index lens 11 penetrates the extending lens support pipe 24 to sink deep into the mouse's brain to reach the deep brain regions thereof.

The present invention is advantageous in that the extending lens support pipe 24 enhances the adhesion stability and installation quality of the miniaturized gradient-index lens 11 to ensure a strong lens-base bond. The present invention meets the need for precision of microstructures by restricting the length of the extending lens support pipe 24 to a range of 2 mm to prevent visual obstructions during the process of inserting the miniaturized gradient-index lens 11 into the mouse's brain. Therefore, the present invention not only enhances optical system stability but also ensures the reliability and efficacy of the miniature modular detachable image-observation base device 100 in miniaturization applications.

In a preferred specific embodiment of the present invention, a plurality of slots 25 are disposed on a lateral side of the wearable optical base 2 and configured to be pressed by a human hand to facilitate the human hand's gripping the slots 25 to increase points of force application in the course of removing the wearable optical base 2 to facilitate the removal of the wearable optical base 2, so as to ensure the convenience and ergonomic handedness of the removal of the wearable optical base 2.

In a preferred specific embodiment of the present invention, the miniature modular detachable image-observation base device 100 further comprises a pulsed laser scanning module that functions as a miniaturized non-linear microscope for achieving micron-scale resolution.

In a preferred specific embodiment of the present invention, the miniaturized non-linear microscope is a miniaturized two-photon microscope.

The present invention is dedicated to neuroscience research design and provides a technology of enabling high-resolution observations of neurofunctional images of deep brain regions. The technology, combined with proprietary optomechanical design, precisely aligned non-linear optical systems, and innovative electronic and software integration, entails placing a high-precision composite lens unit in a wearable optical base with a curved surface that matches the natural curvature of a mouse's skull, and firmly fixing the composite lens unit to the mouse's head. The device overcomes the depth limitations of conventional microscope technology such that researchers can conduct steady, prolonged observations of neuronal activity in the deep brain regions, conduct the functional imaging monitoring of neuronal activity over prolonged periods, and thereby conduct an in-depth study of the functions and pathology of these important brain regions. Details of the design and technology of the present invention are presented below.
1. Composite lens configuration and miniaturized gradient-index lens application: The present invention provides a miniature modular detachable image-observation base device comprising a composite lens unit. The composite lens unit comprises a miniaturized gradient-index lens and a micro coupling lens. The composite lens unit can be replaced with others to accommodate observation applications at different depths to not only overcome limitations of shallow brain regions but also provide the miniaturized gradient-index lens being slender and thus able to pierce the mouse's brain to reach deep brain regions (for example, 4.3 mm in depth) thereof in order for neuronal activity in the deep brain regions to be observed. The selection of the miniaturized gradient-index lens is crucial for minimizing its negative effects on the mouse's health and increasing its survival rate. This can be achieved by ensuring that its length meets depth observation requirements while keeping its diameter as small as possible.
2. Miniaturized design: Miniaturize the entire microscopic imaging system and design a lightweight (weighing less than 1 gram) wearable optical base that can be worn on the mouse's head for extended periods. This ensures that the device does not interfere with the mouse's natural behavior during prolonged experiments while maintaining stable observational performance. The wearable optical base features a special design and thus overcomes the depth limitations of prior art. The special design of the wearable optical base has features as follows:
2-1. A curved surface of the wearable optical base matches the curvature of the mouse's skull: The composite lens unit is placed in the wearable optical base, and the wearable optical base fits the mouse's skull perfectly because the curved surface of the wearable optical base matches the curvature of the mouse's skull. This design enhances the stability of the device, reduces the impact on the mouse's normal behavior, and enables extended functional imaging monitoring of neuronal activity, making it especially suitable for long-duration, continuous neural imaging research.
2-2. Use of screw fixation technology: Screw fixation technology is used to ensure the stability of the device in prolonged experiments. The wearable optical base is firmly fixed to the mouse's skull with screws and thus is firmly fixed in place during the prolonged experiments to ensure the reliability of image data.
3. Non-linear optical system: High-resolution (micron-scale resolution) observations of deep brain region images are carried out with non-linear optical laser scanning technology, such as two-photon microscopy imaging.
4. Magnetic attachment design: Develop an innovative magnetic attachment system conducive to quick mounting and unmounting to dispense with any intricate adjustment process. This structural design greatly simplifies experiment preparation work and reduces discomfort in the course of the experiments.
5. Proprietary optomechanical and electronic design: Precisely designed optomechanical systems ensure the optimal alignment of non-linear optical components as well as the proprietary electronic and software control system integration to achieve synchronous and stable imaging. These structural designs ensure that an imaging system can operate steadily in a high-resolution condition and capture neurofunctional images of deep brain regions (4.3 mm in depth).

The breakthrough enabled by this technology of the present invention lies in its ability to achieve stable high-resolution imaging of deep brain regions without compromising the survival rate of the mouse, so as to provide neuroscience research with a new tool and assist with the exploration of the neural mechanism of deep brain regions.

Therefore, the advantages of the technology of the present invention are as follows:

Capability of performing observations of enhanced depth: The present invention overcomes the depth limitations of prior art such that researchers can clearly observe and analyze neurofunctional images of deep brain regions.

High resolution: The present invention, coupled with non-linear optical laser scanning technology, achieves micron-scale resolution and thus can provide images of higher precision than prior art to enhance the accuracy in neural activity observation and the ability to capture details thereof.

Sustained and stable observations: Compared with prior art, the present invention provides a lightweight design (weighing less than 1 gram) and a secure fixation mechanism to allow for prolonged, stable attachment to the mouse's head, supporting multiple repeated experiments, reducing disturbance to the animal, and enhancing the continuity and reproducibility of experimental data.

Quick experiment preparation: The present invention uses a magnetic attachment design, reducing experiment preparation time significantly, enhancing research efficiency, and reducing operational complexity.

The following examples are provided merely to illustrate the details and concepts of the present invention but are not intended to limit the scope of the claims of the present invention.

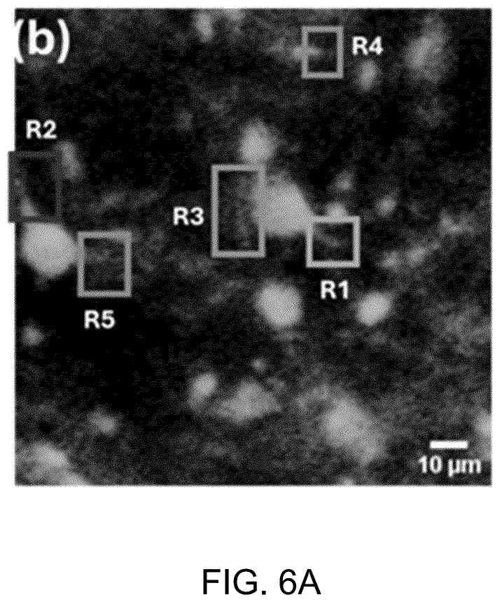

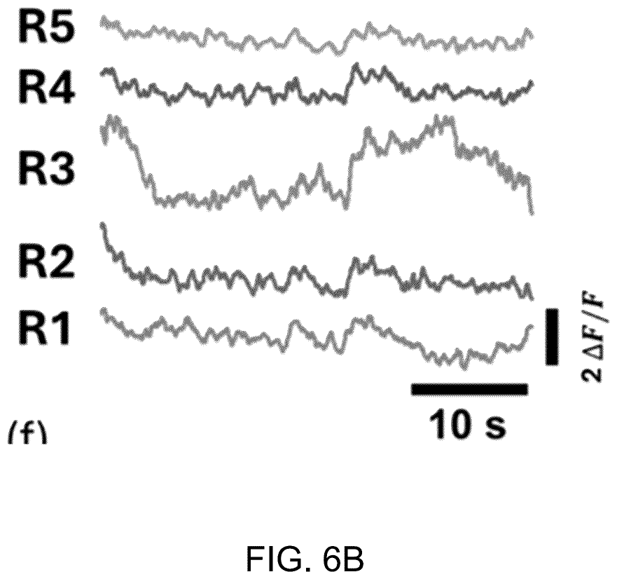

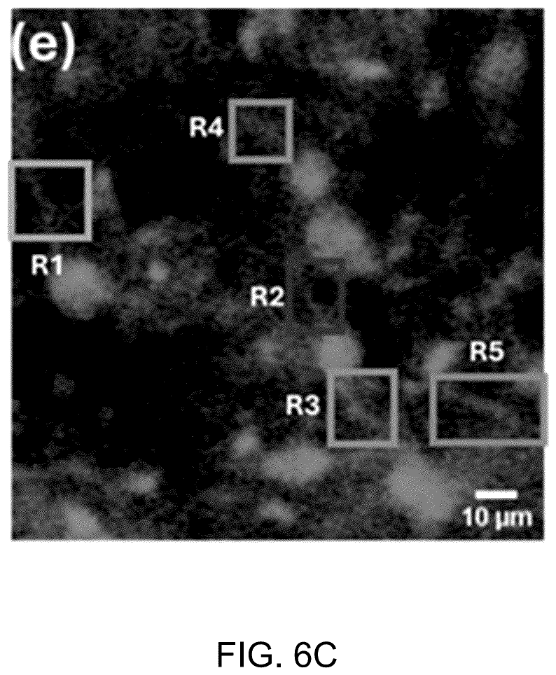

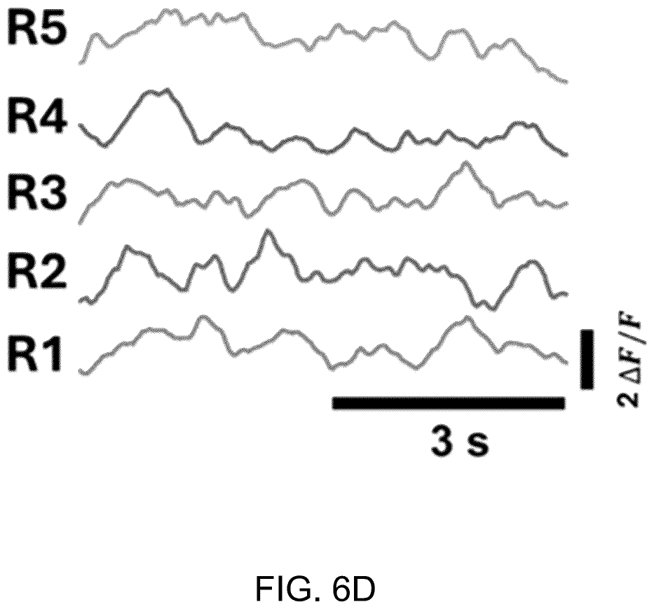

FIG. 5A~5H and FIG. 6A~6F illustrate specific applications of the present invention and show that the wearable optical base of the present invention is effective in observing neurofunctional images of the mouse's deep brain regions (4.3 mm in depth) as well as calcium images generated from the resultant dendrite neurofunctional images of the deep brain regions and successfully obtaining calcium curves through analysis to serve diagnosis purposes. Related details are further presented below.

Images of the mouse wearing the wearable optical base of the present invention in sleep mode and in movement mode are shown in FIG. 5A and FIG. 5E respectively. Neurofunctional images of deep brain regions (4.3 mm in depth) of the mouse in sleep mode are generated through optical design and software development according to the present invention and shown in FIG. 5B and FIG. 5C, respectively. Neurofunctional images of deep brain regions (4.3 mm in depth) of the mouse in movement mode are generated through optical design and software development according to the present invention and shown in FIG. 5F and FIG. 5G, respectively. Curves of calcium images generated from the neurofunctional images through neurofunctional imaging analysis performed on the mouse in sleep mode and movement mode over a long-time scale according to the present invention are shown in FIG. 5D and FIG. 5H, respectively.

The images of the mouse wearing the wearable optical base of the present invention in sleep mode and in movement mode are shown in FIG. 5A and FIG. 5E respectively. Dendrite neurofunctional images of deep brain regions (4.3 mm in depth) of the mouse in sleep mode and movement mode are generated through optical design and software development according to the present invention and shown in FIG. 6A and FIG. 6C, respectively. Curves of calcium images generated from the dendrite neurofunctional images over a long-time scale are shown in FIG. 6B and FIG. 6D. The aforesaid diagrams are conducive to understanding symptoms associated with neurological disorder.

In conclusion, the present invention is a miniature modular detachable image-observation base device effective in overcoming drawbacks of prior art. The device is fixed to a mouse's head and configured to observe neuronal activity in the mouse's deep brain regions and behavior over prolonged periods. The device records the mouse's brain neuronal activity in a way characterized by stability and high resolution, is especially applicable to neurobiology, behavioral neuroscience and brain disease research, assists drug developers in evaluating the effects and potential side effects of drugs on the brain, speeds up drug testing and validation process significantly, allowing the present invention to be innovative and practical and meet user needs.

The preferred embodiments herein disclosed are not intended to unnecessarily limit the scope of the invention. Therefore, simple modifications or variations belonging to the equivalent of the scope of the claims and the instructions disclosed herein for a patent are all within the scope of the present invention.

What is claimed is:

1. A miniature modular detachable image-observation base device, configured for use in non-linear optical high-resolution neuroimaging monitoring, comprising:
a composite lens unit comprising a miniaturized gradient-index lens and a micro coupling lens; and
a wearable optical base having a lower edge, the lower edge having a curved surface that matches a natural curvature of a mouse's skull, with magnetic holes disposed on an upper side of the wearable optical base and configured to engagingly receive magnets to allow the magnets to be coupled to the wearable optical base and fixed firmly thereto by magnetic attraction, the magnets corresponding in number to the magnetic holes, with the composite lens unit detachably mounted inside the wearable optical base to allow the composite lens unit to be firmly fixed to the mouse's head through the wearable optical base, the composite lens unit being replaceable with others to accommodate observation applications at different depths to not only overcome limitations of shallow brain regions but also provide the miniaturized gradient-index lens being slender and thus able to pierce the mouse's brain to reach deep brain regions thereof, so as to steadily perform functional imaging monitoring of neuronal activity in the deep brain regions during numerous sessions over prolonged periods.

2. The miniature modular detachable image-observation base device according to claim 1, being less than 1 gram in weight.

3. The miniature modular detachable image-observation base device according to claim 1, wherein a screw mounting sleeve is disposed on a lateral side of the wearable optical base, allowing a screw to be mounted on the screw mounting sleeve to secure and align the wearable optical base.

4. The miniature modular detachable image-observation base device according to claim 1, wherein a plurality of fixation points are disposed on an upper portion of the wearable optical base to hold and secure a material for filling pores, receive screws for functioning as the fixation points in order to be fixed to the mouse's skull, and receive a filling material for enhancing fixation and reducing slight movement.

5. The miniature modular detachable image-observation base device according to claim 4, wherein the filling material consists of a dental material and a biocompatible colloid.

6. The miniature modular detachable image-observation base device according to claim 1, wherein the wearable optical base has therein a lens receiving chamber for receiving the micro coupling lens, and the lens receiving chamber is in communication with an extending lens support pipe, allowing the miniaturized gradient-index lens to penetrate the extending lens support pipe to sink deep into the mouse's brain to reach the deep brain regions thereof.

7. The miniature modular detachable image-observation base device according to claim 6, wherein the extending lens support pipe is less than 2 mm in length.

8. The miniature modular detachable image-observation base device according to claim 1, further comprising a pulsed laser scanning module for functioning as a miniaturized non-linear microscope configured to perform micron-scale resolution functional imaging monitoring of the deep brain regions.

9. The miniature modular detachable image-observation base device according to claim 8, wherein the miniaturized non-linear microscope is a miniaturized two-photon microscope.

10. The miniature modular detachable image-observation base device according to claim 1, wherein a plurality of slots are disposed on a lateral side of the wearable optical base and configured to be pressed by a human hand to facilitate the human hand's gripping the slots to increase points of force application during removal of the wearable optical base to facilitate the removal of the wearable optical base.
